(19) United States
(12) Patent Application Publication
(10) Pub. No.: US 2025/0295529 A1
(43) Pub. Date: Sep. 25, 2025

(54) DISPOSABLE PAD WITH A POCKET

(71) Applicants: Adam Gorcey, Eatontown, NJ (US); Steven Gorcey, Eatontown, NJ (US)

(72) Inventors: Adam Gorcey, Eatontown, NJ (US); Steven Gorcey, Eatontown, NJ (US)

(21) Appl. No.: 18/614,647

(22) Filed: Mar. 23, 2024

(51) Int. Cl. A61F 13/45

(52) U.S. Cl. CPC A61F 13/45

(57) ABSTRACT

A pad, such as a disposable pad, has a first portion and a second portion. The second portion is formed into a pocket and is movable with respect to the first portion. The first portion can have various arrangements of layers, including absorbent and waterproof layers. An interior of the pocket can be at least partially surrounded by a waterproof layer to hold fluid therein. Adhesive portions can optionally be provided on a bottom side of the first portion for removably securing the first portion to a surface, such as a hospital bed, table or other surface. A supporting member can be positioned between the first portion and the second portion to aid in maintaining the pocket in an open position.

### BACKGROUND

Preventing or limiting fluids from seeping or traveling through a cloth or pad is needed in various industries. One such industry is the healthcare industry where fluids from patients may frequently escape or otherwise move to undesirable locations. As a result, cloths or pads are generally provided adjacent a patient to prevent or limit the movement of fluids escaping from a patient. Frequently, the patient is positioned on a bed with a cloth or pad positioned under the patient. While the pad or cloth may prevent fluid from traveling through it into the bed, there is nothing preventing fluid from moving along the pad or cloth and over an end or side of the table or bed. In such a case, a healthcare worker positioned about the bed can come into contact with the fluids.

In other situations, the cloth or pad may become fully saturated by fluid and fluid may travel, especially if the cloth or pad is under the patient, in a direction toward the ends or side of a bed. This is a frequent problem when a patient moves on a saturated pad or cloth. As a result, fluid can travel across the bed and onto the healthcare worker and/or the floor creating a safety and health risk.

One specific example of a disposable cloth or pad that is used in the healthcare industry is a hospital Chux pad. Chux pads came from the fact that these thin pads were simply “chucked” in the trash after use. The hospital chuck generally has an absorbent layer and can be positioned under the patient to capture escaping fluids from the patient. Depending on the position of the patient, fluid can be accidentally directed along the pad and toward an end of the bed of such patient. As the hospital chucks become saturated, fluid can be forced by the force of the patient against the bed laterally in a direction toward the end or sides of the bed. The fluid can then accidentally spill onto a healthcare provider or the floor. Obviously, such situations are not desirable and a need exists for an apparatus capable of limiting or remedying one or more of these situations.

### DRAWINGS

The following descriptions are provided for reference and are not intended to limit the disclosure to only the embodiments shown.

FIG. 1 illustrates a top side of an apparatus having one or more layers and a pocket formed on one end in an embodiment of the present disclosure.

FIG. 2 illustrates a bottom side of an apparatus having one or more layers and a pocket formed on one end in an embodiment of the present disclosure.

FIG. 3 illustrates a plurality layers of a first portion or a second portion of an apparatus of in an embodiment of the present disclosure.

FIG. 4 illustrates an apparatus having one or more layers and a pocket positioned hanging over an end of a table in an embodiment of the present disclosure.

FIG. 5 illustrates a top side of an apparatus having a pocket and one or more adhesive portions in an embodiment of the present disclosure.

FIG. 6 illustrates an apparatus having one or more layers and a pocket with a support portion positioned hanging over an end of a surface in an embodiment of the present disclosure.

FIG. 7 illustrates an apparatus having one or more layers, a pocket and a splash guard in an embodiment of the present disclosure.

FIG. 8 illustrates an apparatus having an aperture for a medical device in an embodiment of the present disclosure.

### DETAILED DESCRIPTION

Characteristics and advantages of the present disclosure and additional features and benefits will be readily apparent to those skilled in the art upon consideration of the following detailed description of exemplary embodiments and/or referring to the accompanying FIGures. It should be understood that the description herein and appended drawings, being of example embodiments, are not intended to limit the claims of this patent or any patent or patent application claiming priority hereto. On the contrary, the intention is to cover all modifications, equivalents and alternatives falling within the spirit and scope of this disclosure and the appended claims. Many changes may be made to the particular embodiments and details disclosed herein without departing from such spirit and scope.

In showing and describing embodiments of the present disclosure in the appended figures, common or similar elements are referenced with like or identical reference numerals or are apparent from the figures and/or the description herein. The figures are not necessarily to scale, and certain features and certain views of the figures may be shown exaggerated in scale or in schematic in the interest of clarity and conciseness.

As used herein and throughout various portions (and headings) of this patent (including the claims), the terms “invention”, “disclosure”, “present invention”, “present disclosure”, and variations thereof are not intended to mean every possible embodiment encompassed by this disclosure or any particular claim(s). Thus, the subject matter of each such reference should not be considered as necessary for, or part of, every embodiment hereof or of any particular claim(s) merely because of such reference.

Certain terms are used herein and in the appended claims to refer to particular components. As one skilled in the art will appreciate, different persons may refer to a component by different names. Also, the terms “including” and “comprising” are used herein and in the appended claims in an open-ended fashion, and thus should be interpreted to mean “including, but not limited to . . . ”. Further, reference herein and in the appended claims to components, feature, actions, aspects, etc. in a singular tense does not limit the present disclosure or appended claims to only one such component feature, action, aspect, etc., but should be interpreted to mean one or more, except and only to the extent as may be expressly limited otherwise herein or in a particular claim hereof and only for such claim(s) and any claim(s) depending therefrom

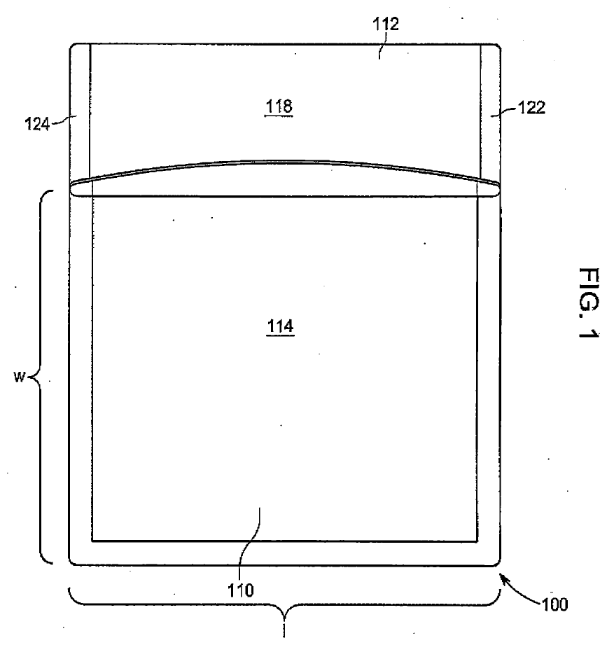

FIG. 1 illustrate an apparatus 100 in an embodiment of the present disclosure. The apparatus 100 may generally be referred to as a pad 100. The pad 100 may be a reusable or a one-time use disposable type pad. While generally shown rectangular in shape, the pad 100 should not be limited to such shape and may be any geometric shape as desired, including but not limited to square, circular, oval, elliptical, hexagonal, octagonal, or other shape corresponding to a desire of a user of the pad 100.

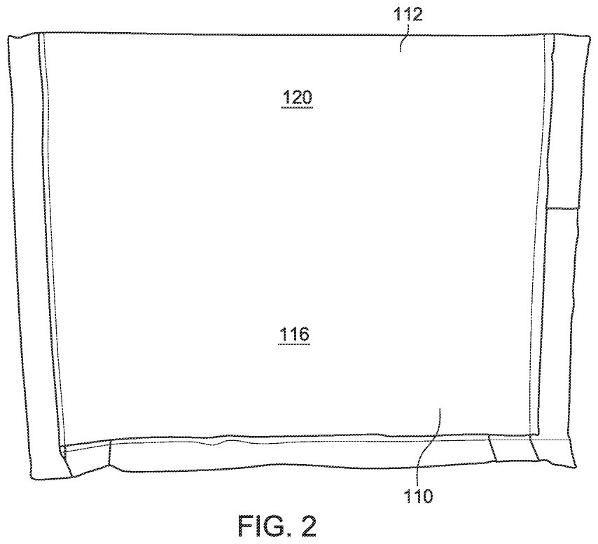

The pad 100 may have a first portion 110 and a second portion 112. The first portion 110 may have a length, l, and a width, w, defining an area of the first portion 110 of the pad 100. The first portion 110 may have a top side 114, and a bottom side 116 opposite the top side 114 as shown in FIG. 2. The area of the first portion 110 may be greater than the area of the second portion 112.

The second portion 112 may have a first side 118 and a second side 120. The first side 118 can be integrally formed with the second side 120 or may be attached or secured to provide a fluid tight seal between the first side 118 and the second side 120. In an embodiment, the first side 118 and the second side can be integrally formed with the first portion 110. The second portion 112 is movable with respect to the first portion 110. For example, the second portion 112 may be folded toward the first portion, such as by folding the first side 118 onto the second side 120. A third side 122 and a fourth side 124 can be positioned between and/or connect the first side 118 and the second side 120. The third side 122 and the fourth side 124 can provide a fluid tight seal between the first side 118 and 120.

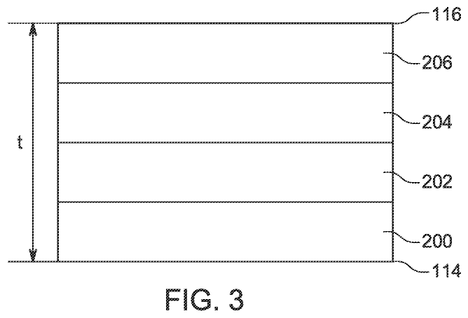

A thickness, t, defined between the top side 114 and the bottom side 116 is shown in FIG. 3. In an embodiment, the first side 118 and the second side 120 can each have a thickness substantially similar to and/or identical to the thickness t. In such an embodiment, the total thickness of the first side 118 and the second side 120 can be about twice the thickness t.

The thickness t can be defined by and/or comprised of one or more layers of material. For example, the first portion 110 and/or the second portion 112 may have an absorbent layer comprised of one or more types of material that absorbs fluids. As a non-limiting example, the absorbent material can comprise a nonwoven material, cellulose pulp, polyester, viscose, cotton, a hydrophilic homopolymer or copolymer material, such as a superabsorbent polymer material, combinations thereof, and/or functionally similar material. The first portion 110 and/or the second portion 112 may comprise a waterproof layer that may be made of any type of material that traps and/or prevents fluid from moving therethrough. As a non-limiting example, the waterproof layer can be made of a polyurethane, laminate, polyurethane laminate, thermoplastic, thermoplastic polyurethane, or the like.

The first portion 110 and/or the second portion 112 can comprise an adhesive layer to adhere the pad 100 to one or more surfaces. The adhesive material may be used to secure the first portion 110 to the second portion 112. The adhesive material may be used to secure the first side 118 to the second side 120. As will be later described, the adhesive layer can be used to secure the bottom side 116 to a surface, such as but not limited to a bed, table, operating table, or hospital bed.

The adhesive material may comprise a material generally used in the diapering industry to adhere or removably adhere, the pad to a surface, such as a bed or table. As a non-limiting example, the material used may be the same material used to make straps used to secure a diaper around a baby and onto itself, a Velcro, a fastening and release tap or other substance or device capable of removably securing the first portion 110 to a surface.

FIG. 3 illustrates an embodiment showing a cross-section of the first portion 110 or the second portion 112. For example, the thickness t of FIG. 3 can comprise a cross-section of the first side 118 and/or the second side 120 of the second portion 112. The first portion 110 may have a similar or different cross-section from the second portion 112. In addition, sections of the first portion 110 may have different cross-sections than other sections of the first portion 110.

The first side 118 can have the same number and arrangement of layers as the second side 120 or different depending on the application and desire of the user. In an embodiment where the pad 100 has an integrally formed first portion 110 and second portion 112, the layers of the first portion 110 and second portion 112 may be the same. The layers of the first side 118 and the second side 120 may have the same number of layers.

While various numbers of layers are contemplated, the embodiment of FIG. 3 illustrates a first layer 200, a second layer 202, a third layer 204 and a fourth layer 206. In such an embodiment, the first layer 200 may be adjacent to the top side 114, and the fourth layer 206 may be adjacent the bottom side 116. Various embodiments and arrangements of these layers are contemplated in the present disclosure. As will be appreciated by a person having ordinary skill in the art, the number and arrangement of the layers may be adjusted or changed depending on the application of the pad 100 and other considerations or factors relevant to a user of the pad 100.

Accordingly, in one example the first layer 200 is an absorbent material, the second layer 202 is an absorbent material, the third layer 204 is a waterproof material, and the fourth layer 206 is an adhesive material. The absorbent material of the first layer 200 may be the same or a different type of absorbent material from the second layer 202. For example, the first layer 200 may be a non-woven absorbent material, and the second layer 202 may be a superabsorbent polymer material. In another example, the first layer 200 and the second layer 202 can be absorbent material, such as paper or a cellulose pulp, the third layer 204 may be a waterproof layer, and the fourth layer 206 may not be included. In yet another example, the first layer 200 may be an absorbent material, the second layer 202 may be a different absorbent material, the third layer 204 may be a waterproof layer, and the fourth layer 206 may be an adhesive layer. These examples should not be limiting of the present disclosure, and a person having ordinary skill in the art will appreciate that the number, size and arrangement of the layers may be modified depending on the application and need of a user.

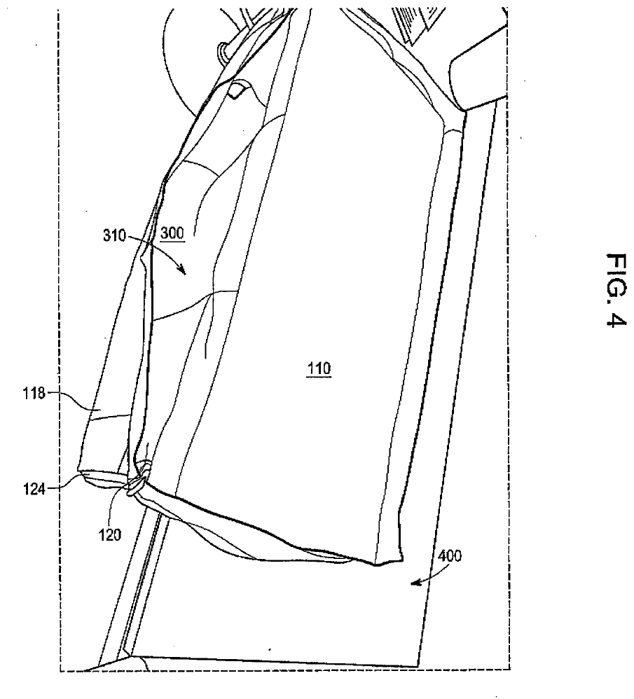

As shown in FIG. 4, the first portion 110 may be movable with respect to the second portion 112. In an embodiment, the first portion 110 may be positioned on a bed, table or other surface, and the second portion 112 may extend over an edge of such bed, table or other surface. For example, the second portion 112 may be movable to a position such that the second portion 112 is substantially perpendicular to the first portion 110. The second portion 112 may be separate from the first portion 110 or integrally formed therewith. If the second portion 112 is separate from the first portion 110, then the second portion 112 may be secured to the first portion 110. For example, an adhesive may be used to secure the second portion 112 to the first portion 110. In an embodiment, the second portion 112 may be detachable from the first portion 110.

The second portion 112 may be fluidly sealed to form a pocket 300. The pocket 300 may be formed in various manners. For example, the second portion 112 may have the first side 118 secured to the second side 120 to form the pocket 300. The pocket 30 may be formed by securing the first side 118 to the second side 120 with the third side 122 and the fourth side 124. In an embodiment, the first side 118 can be integrally formed with the second side 120.

The opening 310 can be positioned at one end of the pocket 300. The interior of the pocket 300 can provide a space where fluid and/or other substances can be trapped between the sides 118, 120, 122, and 124. In an embodiment, the interior of the pocket 300 can be lined with an absorbent material as described above. The first side 118 and the second side 120 may each have the thickness t and be arranged such that the absorbent material layer faces an interior of the pocket 300. The waterproof material layer may at least partially surround the interior of the pocket 300 and can trap or otherwise hold fluid therein. In an embodiment, the waterproof material layer can be positioned on the external surface of the pocket 300 to trap the fluid between the interior and the waterproof layers of the first side 118 and the second side 302. To this end, the waterproof layer may be positioned within the sides 118, 120. The pocket 300 can form a sort of bowl like structure capable of holding liquid fluids as well as other substances therein.

In use, the first portion 110 of the pad 100 may be positioned on a surface 400, as shown in FIG. 4. As an example, the first portion 110 can be positioned on the surface 400 such that the second portion 112 extends beyond an edge of the surface 400. As a non-limiting example of use of the pad 100, a patient can be positioned such that the first portion 110 of the pad 100 is positioned between the patient and the surface 400. The first portion 110 may be removably adhered or otherwise removably secured to the surface 400 such that one or more waterproof layers are adjacent the surface 400, and an absorbent material is positioned opposite the waterproof layers and/or adjacent to the patient.

The second portion 112 is movable with respect to the first portion 110. The second portion can be moved such as by folding the second portion 112 with respect to the first portion 110. The second portion 112 may be positioned at an angle with respect to the first portion 110 and the surface 400. Where the surface 400 has a ledge or edge, the second portion 112 can be moved to conform to the end of the surface 400 and extend downward therefrom, as shown in FIG. 4. The second portion 112 can be movable at any angle along a substantially 360 degree path. In an embodiment, the second portion 112 can be moved such that the second portion 112 is substantially perpendicular to the first portion 110.

The first portion 110 can be removably secured to the surface 400 such that the pocket 300 has an opening 310 adjacent the first portion 110 as shown in FIG. 4. The opening 310 can extend along the length l of the first portion. For example, the pocket 300 can extend along the length l of the first portion such that the interior of the pocket 300 is accessible along a substantial portion of the length l. The absorbent material layer may be within the pocket 300 and facing and/or in contact with the interior of the pocket 300. Fluids or other material entering the pocket 300 may first contact the absorbent material layer within the pocket 300. The waterproof material layer may be external to the absorbent material layer and substantially surround the interior of the pocket 300.

In the event a bodily fluid, medication, or other substance spills on or otherwise moves to the pad 100, all or a portion of the fluid may be absorbed by one or more absorbent layers of the pad 100. The pad 100 can become saturated and the force or weight of the patient may force fluid toward the pocket 300. The fluids could instead spill or otherwise move quickly toward the pocket 300 without becoming totally absorbed. In these and other situations, the fluid from the pad 100 may travel along the first portion 110 and through the opening 310 into the interior of the pocket 300 of the second portion 112. If so, the fluid can be captured in the pocket 300 and prevented from traveling out of the pocket 300, such as toward the ground. For example, the interior of the pocket 300 may then absorb the fluids into the absorbent material layer. The waterproof material layer of the second portion 112 can prevent the fluid from escaping the pocket 300. In many instances, if healthcare workers or other individuals are standing about the surface 400, the pocket 300 can capture fluids before coming into contact with such healthcare workers or other individuals. Without the pocket 300, fluid or other substances may travel along the first portion 110 and over an edge of the bed or table and onto the healthcare worker, such as the feet of the healthcare worker or on the floor about the table or bed creating a hazard.

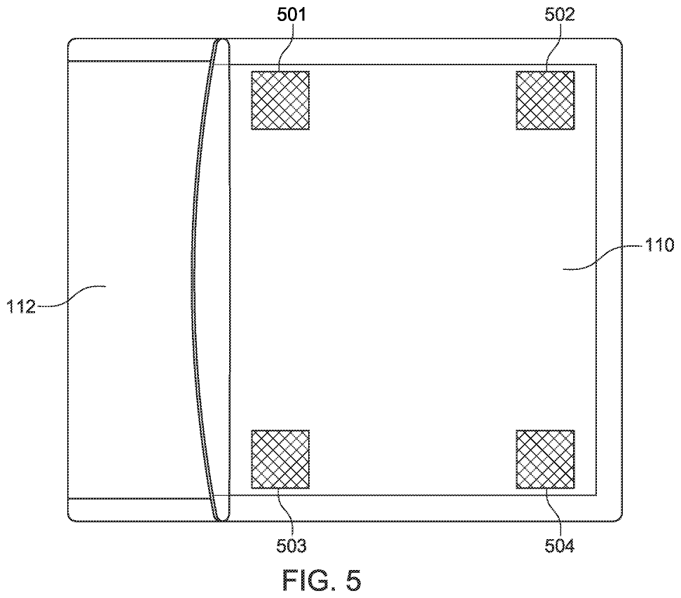

FIG. 5 illustrates an embodiment of the pad 100 having adhesive portions 500-504 positioned about the first portion 110. The adhesive portions 500-504 can be positioned on the bottom side 116 of the pad 100. The adhesive portions 500-504 may generally be positioned about the perimeter of the first portion 110 to secure or removably attach the first portion 110 to the surface 400. While not generally shown, the adhesive portions 500-504 may extend continuously around the perimeter of the first portion 110.

The adhesive portions 500-504 can be made of any type of material or substance to secure or removably attach the first portion 110 to the surface 400 or other surface desired by a user. For example, the adhesive portions 500-504 may comprise any of the materials and substances described above with respect to the adhesive material layer.

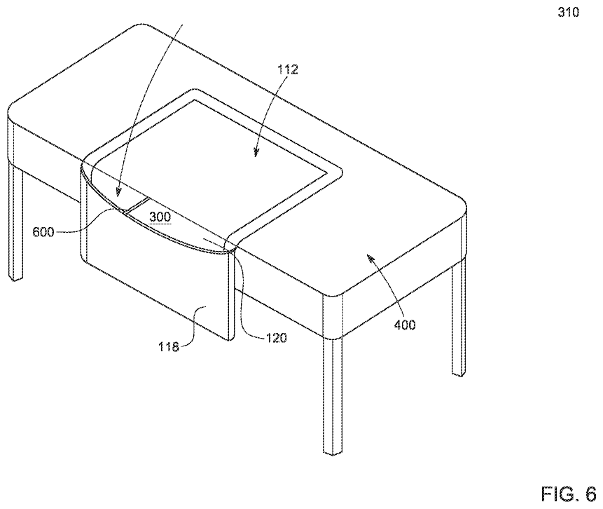

FIG. 6 illustrates another embodiment of the pad 100 where a supporting portion 600 is positioned adjacent the pocket 300. The supporting portion 600 may be extend along a top of the pocket 100 to maintain or at least aid in keeping the interior of the pocket 300 open to receive any fluids or other substances from the pad 100. The supporting portion 600 may extend from a position substantially in the middle of the pocket 300 or about half the length l of the first portion 110. Various arrangements of the supporting portion 600 are contemplated herein. For example, the supporting portion 600 can comprise multiple portions, various sizes and/or various materials positioned between the first portion 110 and the second portion 112.

The supporting portion 600 can comprise the absorbent material layer and the waterproof layer. The supporting portion 600 can comprise less layers of material than the first portion 110 and the second portion 112. The absorbent material layer and the waterproof material layer may extend from the first portion to form the supporting portion 600. The supporting portion 600 may comprise a material with some resilience to maintain the accessibility of the interior of the pocket 300. The material and structure of the supporting portion 112 can be arranged such that the second portion 112 biases the pocket 300 to an open position.

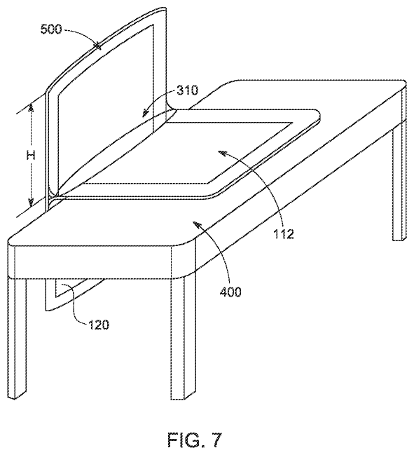

In FIG. 7, another embodiment of the present disclosure includes a splash guard 500. The pad 100 has a second portion 112 position on or about the surface 400. The opening 310 of the pocket 300 is positioned between the splash guard 500 and the second portion 112. The splash guard 500 can extend vertically from the table and/or substantially perpendicular to the second portion 112. The outer surface or first side 118 of the pocket 300 can extend vertically the height H to form the splash guard 500. To this end, the splash guard 500 can be formed of any of the aforementioned materials of the pad 100. In one or more embodiments, the splash guard 500 is formed of or comprises material having a resiliency that may permit the splash guard 500 to remain vertical with respect to the second portion 112. The splash guard 500 can advantageously prevent fluid from moving away from the surface 400 and splashing or otherwise moving with a vertical component with respect to the table 400.

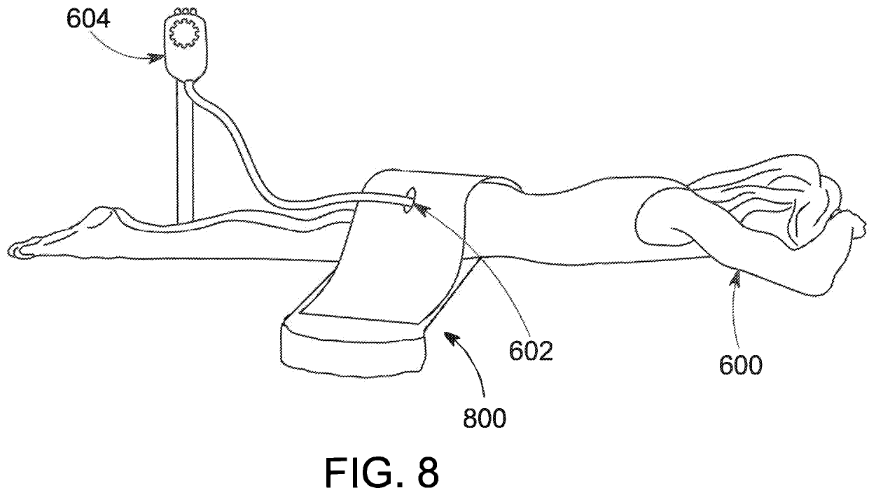

FIG. 8 illustrates yet another embodiment of the present disclosure. In this embodiment, a pad 800 comprises a sheet or mat that may be placed on top of or over a patient 600. The pad 800 may have some or all of the features of the pad 100 herein described. For example, the pad 800 may be comprised of one or more absorbent layers and/or adhesive layers as previously described. The pad 800 may be used to further protect an individual from material spilling or otherwise escaping the patient 600. A medical professional, for example, may place the pad 800 over the patient to serve as a barrier to bodily fluids from the patient contacting the medical professional. The pad 800 may further comprise an aperture 602 extending through the pad 800 and permitting insertion of a device therethrough. In an embodiment, a medical device 604, such as a colonoscope, is shown inserted through the pad 800 in FIG. 8. Advantageously, the pad 800 provides a fluid barrier for the medical professional while permitting the medical device 604 to pass through the aperture 602 and into the patient 600. Accordingly, the pad 800 may be positioned between the medical professional or other user and the patient 600.

As a mere example for explanation purposes, the pad 100 or the pad 800 may comprise the first portion 110 and the second portion 112 integrally formed thereof. A user can fold the first portion 110 onto itself toward the first portion to form the pocket 300. The first side 118 can be secured to the second side 120. The user can adhere the first side 118 to the second side 120 of the second portion 112 with use of an adhesive layer positioned therebetween. In another embodiment, the pocket 300 may come pre-formed prior to distribution to the user. In any event, the pocket 300 may be formed such that the layers of the first portion 110 are the same as the layers of material in the first side 118 and the second side 120 of the second portion 112. The multi-layered material of the first portion 110 can comprise a waterproof layer adjacent the bottom side 116 and an absorbent material adjacent the top side. As such, the waterproof layer may be the exterior layer of the pocket 300 and absorbent layers of the first side 118 and the second side 120. The user can remove, peel away or release tape from the adhesive portion 500-504 and position the bottom side 116 of the first portion 110 on the surface 400. The user can position the second portion 112 such that it is hanging over an edge of the surface 400. A patient may be positioned by the user on the first portion 110 of the pad 100 such that the pad 100 is positioned between the patient and the surface 400. The user can manipulate the supporting portion 600 to force the pocket 300 open to accept fluids or other substances. At this position, any fluids from or used on the patient can preferably be trapped within the first portion 110 or the second portion 112. Specifically, fluids not capable of being absorbed by the first portion 110 for whatever reason can be trapped in the pocket 300 and prevented from contacting the user and the floor beneath the surface 400.

As used herein, the terms “about” and “substantially” are intended to include the degree of error associated with measurement of the particular quantity based upon the equipment available at the time of filing the application. For example, “about” may include a range of #10%, or 5%, or 2% of a given value or other percentage change as will be appreciated by those of skill in the art for the particular measurement and/or dimensions referred to herein.

The terminology used herein is for the purpose of describing particular embodiments only and is not intended to be limiting of the present disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an illustrative embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

1. A pad comprising:
a first portion having a plurality of layers between a top side and a bottom side, the plurality of layers comprising at least a waterproof layer and an absorbent layer; and
a second portion secured to the first portion, the second portion having a plurality of sides forming a pocket having an interior therein and an opening adjacent the first portion wherein the second portion is movable with respect to the first portion.

2. The pad of claim 1 wherein the second portion is movable to a position such that the first portion is substantially perpendicular to the second portion.

3. The pad of claim 2 wherein the second portion has at least one side having a thickness substantially similar to the thickness of the first portion.

4. The pad of claim 2 further comprising:
one or more adhesive portions to removably secure a bottom side of the pad to a surface.

5. The pad of claim 2 wherein the absorbent material is adjacent the top side and the waterproof layer is adjacent the bottom side.

6. The pad of claim 1 wherein the interior of the pocket is at least partially surrounded by a waterproof layer.

7. The pad of claim 2 further comprising:
a supporting portion positioned between the first portion and the second portion across an opening of the pocket.

8. The pad of claim 2 wherein the supporting portion is comprised of one or more of the plurality of layers of the first portion.

9. The pad of claim 1 wherein first portion is integrally formed with the second portion.

10. The pad of claim 9 wherein the thickness of the first portion is substantially similar to the thickness of one of the plurality of sides of the pocket.

11. A pad comprising:
a first portion having a thickness defined between a top side and a bottom side;
the top side having an absorbent material or a waterproof material; and
a second portion movably secured to the first portion and having a plurality of sides defining a pocket having an interior, wherein the second portion is movable with respect to the first portion.

12. The pad of claim 11 wherein the second portion is movable to a position substantially perpendicular to the first portion.

13. The pad of claim 11 wherein the pocket has at least a waterproof layer surrounding a portion of the interior of the pocket.

14. The pad of claim 11 wherein the first portion has the absorbent material adjacent the top side and the waterproof material adjacent to the bottom side.

15. The pad of claim 11 further comprising a splash guard extending vertically with respect to the first portion or the second portion.

16. The pad of claim 11 further comprising:
adhesive portions on the first portion for removably securing the bottom side of the first portion to a surface.

17. The pad of claim 11 further comprising:
a supporting portion positioned between the first portion and the second portion and aiding in maintaining the pocket in an open position exposing the interior of the pocket.

18. A pad comprising:
a first portion having a thickness defined between a top side and a bottom side, the thickness comprised of a plurality of layers of material wherein at least one of the plurality of layers is an absorbent material and a second layer of the plurality of layers of material is a waterproof layer;
a second portion integrally formed with the first portion, the second portion having at least two sides with each of the two sides having a thickness substantially similar to a thickness of the first portion, the second portion formed into a pocket having an interior therein, and further wherein the pocket is movable with respect to the first portion.

19. The pad of claim 18 further comprising:
adhesive portions on the bottom side of the first portion to secure the first portion to a surface; and
a supporting portion positioned between the first portion and the second portion, the supporting portion comprised of one or more of the plurality of layers of material extending between the first portion and the second portion.

20. The pad of claim 18 wherein the second portion has third and fourth sides fluidly sealed to the first side and the second side to form the interior of the pocket, and further wherein the second portion comprises a waterproof layer about at least a portion of the interior of the pocket.
